(19) United States
(12) Patent Application Publication
(10) Pub. No.: US 2008/0132456 A1
(43) Pub. Date: Jun. 5, 2008

(54) Pharmaceutical Formulation Comprising Low Crystallinity Cellulose

(76) Inventors: Ragnar Ek, Stockholm (SE); Maria Stromme, Uppsala (SE); Albert Mihranyan, Yerevan (AM)

Correspondence Address: DINSMORE & SHOHL, LLP, 1900 CHEMED CENTER, 255 EAST FIFTH STREET, CINCINNATI, OH, 45202, US

(21) Appl. No.: 11/792,217

(22) PCT Filed: Dec. 9, 2005
(86) PCT No.: PCT/IB05/03806

§ 371 Date: Jun. 1, 2007

Related U.S. Application Data
(60) Provisional application No. 60/634,633, filed on Dec. 9, 2004.

(51) Int. Cl. A61K 31/7048; A61K 47/38; A61K 31/21; A61K 31/435; A61K 31/551; A61K 31/19; A61K 31/164; A61K 31/515; A61K 31/43; A61K 31/352

(52) U.S. Cl. 514/29; 514/781; 514/506; 514/192; 514/456; 514/290; 514/270; 514/221; 514/628; 514/567

(57) ABSTRACT

A pharmaceutical composition comprises a pharmaceutically effective amount of an active ingredient and low crystallinity cellulose having a crystallinity index of less than about 75% as measured by X-ray diffraction. The compositions advantageously may be provided in tablet form or granulate form and provide good stability against moisture degradation.

### FIELD OF THE INVENTION

The present invention relates to low crystallinity cellulose (LCC) materials and their use in the manufacture of dry pharmaceutical compositions, in particular tablets, comprising moisture-sensitive drugs.

### BACKGROUND OF THE INVENTION

Microcrystalline cellulose (MCC) is commercially available and commonly used by the pharmaceutical industry as a tabletting excipient (e.g., Avicel PH 102 from FMC, Ireland). Powdered cellulose (PC) grades (e.g. Solca-Floc, Edward Mendell Co. Inc., USA, or Elcema, Degussa Ltd., UK) are also used in tabletting. Both MCC and PC are alfa-cellulose products but differ in the respective process of their manufacture: MCC is produced via acidic hydrolysis with mineral acids, whereas PC is produced by mechanical disintegration. For specifications, see European Pharmacopoiea, 5th Edition, 2005, Council of Europe, Strasbourg, pp. 1228-1235. Considerable efforts have been made to improve the physical as well as the functional properties of various pharmaceutical compositions containing MCC, and a number of published studies on modified cellulose materials focus on parameters such as cellulose moisture sorption isotherms, cellulose crystallinity, surface area and pore volume. In multi-component pharmaceutical systems, these parameters can significantly influence the physical and chemical stability of a drug with respect to interactions with a surrounding atmosphere and with other components in the composition.

A number of drugs, like acetylsalicylic acid (ASA) and penicillines, just to mention a few examples, undergo changes upon contact with moisture. Accelerated stability studies on the moisture-induced drug degradation process at elevated temperatures and at various humidity values constitute an important part of the routine program in connection with drug formulation design and testing.

In order to ensure proper product quality, moisture-sensitive drugs are normally formulated together with excipients that minimize the possibility of undesired changes. This is generally achieved according to one of the following procedures:

i) Using Non-Hygroscopic Excipients:

The most obvious approach to avoid undesired hydrolysis in pharmaceutical systems is to formulate the moisture-sensitive drugs together with excipients which do not attract moisture. However, the choice of the excipient should be accomplished so as to not compromise other important pharmaceutical properties, e.g., tablet-forming ability, etc. Lactose monohydrate is one example of such a material used as a diluent in a fairly large number of tablet compositions. Whereas no or only minor decomposition of the hydrolysis-sensitive drugs is observed when formulated together with this sugar, lactose monohydrate is far from being an optimal component with regard to tablet-forming procedures. Compacts of lactose are weaker than compacts of MCC and, whereas MCC is inert and harmless to human when taken orally, regulatory problems as well as ethical problems may be associated with lactose. Examples of such problems include, e.g., lactose intolerance, lactose being a potential bovine spongiform encephalopathy (BSE) virus carrier, lactose being a non-vegetarian dairy product, etc.

ii) Using Extra-Dried Excipients:

Another common approach to avoid hydrolysis of the moisture-sensitive drugs is to decrease the overall moisture content in a drug delivery vehicle by formulating the drug together with extra-dried grades of excipients. Low moisture grades of microcrystalline cellulose (e.g. Avicel PH 112 with 1.5%, wt./wt., moisture content) are commonly used for this purpose. However, a problem noticed with this approach is that extra-dried grades appear to be hygroscopic and, consequently, the moisture content in the formulation rapidly increases upon exposure to moisture in the ambient air, undermining the stability of the product.

The water sorption patterns of cellulose powders from various sources and of varying crystallinity have been studied. In e.g., Mihranyan et al (2004) “Moisture sorption by cellulose powders of varying crystallinity”, Int. J. Pharm., 269 (2), 433-442, it was concluded that at relative humidities below 75%, the moisture content increases with decreased crystallinity indexes.

Several methods to decrease the crystallinity index of cellulose have been described in the literature, and have often been accomplished by depolymerization in the presence of an acid, e.g. hydrochloric acid and/or sulfuric acid or, commonly, phosphoric acid. U.S. Pat. No. 5,417,984 discloses such a phosphoric acid-based process for preparation of low crystallinity cellulose, suitable for use as a direct compression excipient (e.g., binder, disintegrant and/or diluent). Cellulose is reacted with the acid for a controlled period of time followed by separation, isolation and transformation into the desired form. The low crystallinity product which is obtained serves as an excellent disintegrant in tablets because of its interaction with water. The acid-depolymerized, low crystallinity cellulose is suggested for manufacturing tablets containing, e.g., acetaminophen (very slightly soluble in cold water) and Griseofulvin (practically insoluble in water).

A cellulose-based product with reduced crystallinity has also been produced by hydroxide treatment of mercerizing strength, as described by Kumar et al, (2002) “Preparation, characterization, and tabletting properties of a new cellulose-based pharmaceutical aid”, Int. J. Pharm., 235, 129-140. Kumar et al conclude that the product (UICEL) in comparison with Avicel PH-102 not only acts as a binder but also as a highly effective disintegrant.

An additional low crystallinity cellulose (LCC) product, obtained by reaction with ZnCl₂, is disclosed by Mihranyan et al (2004), cited above. The LCC product was obtained by dispersion of microcrystalline cellulose in a ZnCl₂ solution with vigorous stirring. After swelling, additional water was added. The resultant powder was washed repeatedly until the conductivity of the washed water approximated that of deionized water, and thereafter spray-dried. After concluding that moisture in microcrystalline cellulose may cause stability problems for moisture sensitive drugs, the authors describe the aim of their study to investigate the influence of crystallinity index and surface area on the uptake of moisture in cellulose powders. Based on a comparison between low crystallinity cellulose, agglomerated micronized cellulose, microcrystalline cellulose and cellulose from Cladophora and Algiflor algae, they made the conclusion referred to above: namely, that moisture content increases with decreasing crystallinity index. In particular there is no indication whatsoever in the article that a low crystallinity product would be of interest in formulations comprising a moisture sensitive drug. ZnCl₂ has also been used for treatment of cotton cellulose but without any suggestion for manufacture of tablets based on the resulting product (see Patil et al (1965) “Studies on decrystallisation of cotton”, Textile Res. J., June, 517-523).

### SUMMARY OF THE INVENTION

It has now surprisingly been found that low crystallinity cellulose, in spite of the well-documented increased moisture sorption compared to microcrystalline cellulose, is suitable for use in combination with moisture sensitive drugs.

Accordingly, the present invention is directed to pharmaceutical compositions comprising a pharmaceutically effective amount of an active ingredient, i.e., a drug, and low crystallinity cellulose. Within the present specification, low crystallinity cellulose is intended to mean cellulose having a crystallinity lower than that of presently commercially available MCC, and particularly having a crystallinity index of less than about 75% as measured by X-ray diffraction. Advantageously, the active ingredient may comprise a moisture sensitive component, i.e., a moisture-sensitive drug.

The compositions of the invention are advantageous in avoiding undesirable moisture mediated and/or moisture induced degradation, such as e.g. hydrolysis, of active ingredients, in providing stable compositions, and/or in providing compositions which are suitable for tablet formation, granulate formation, and the like. These and additional advantages will be more fully understood in view of the following detailed description.

### BRIEF DESCRIPTION OF THE DRAWINGS

The following detailed description will be more fully understood in view of the drawings in which:

FIG. 1 sets forth schematically the hydrolysis degradation reaction of acetyl salicylic acid (ASA);

FIG. 2 sets forth ASA degradation in terms of measured salicylic acid content of three cellulose-containing compositions after 5-6 weeks of storage;

FIGS. 3A-3D set forth ASA degradation in terms of measured salicylic acid content of various cellulose-containing compositions stored under approximately 11%, 40%, 50% and 75% relative humidity, respectively, as a function of time;

FIG. 4 sets forth ASA degradation in terms of measured salicylic acid content of various cellulose-containing compositions with varying ratios of acetylsalicylic acid and cellulose therein; and

FIG. 5 sets forth schematically a method for producing the low crystallinity cellulose of the present invention by spraying a swelling agent onto a layer of microcrystalline cellulose and then washing, as described in greater detail below.

### DETAILED DESCRIPTION

The pharmaceutical compositions according to the present invention comprise a pharmaceutically effective amount of an active ingredient, i.e., a drug, and low crystallinity cellulose. As will be described in further detail below, the low crystallinity cellulose is prepared, in one embodiment, by swelling ordinary MCC in a solution to decrease the crystallinity of the cellulose. Surprisingly, moisture-sensitive drugs in contact with the low crystallinity cellulose resist degradation. Thus, the low crystallinity cellulose is advantageous for use in the manufacture of dry compositions comprising moisture-sensitive drugs. In particular, it has been found that by producing mixtures of moisture-sensitive drugs with low crystallinity cellulose, undesired hydrolysis of the drug can be effectively avoided. The low crystallinity cellulose is stable and is an excellent tabletting powder and/or excellent granulating additive.

This enables one to produce tablets of moisture-sensitive drugs containing cellulose where effective stability, similar to that for mixtures with lactose monohydrate, is combined with the excellent tabletting characteristics exhibited by ordinary MCC, which can be considered as a standard for desirable tabletting properties.

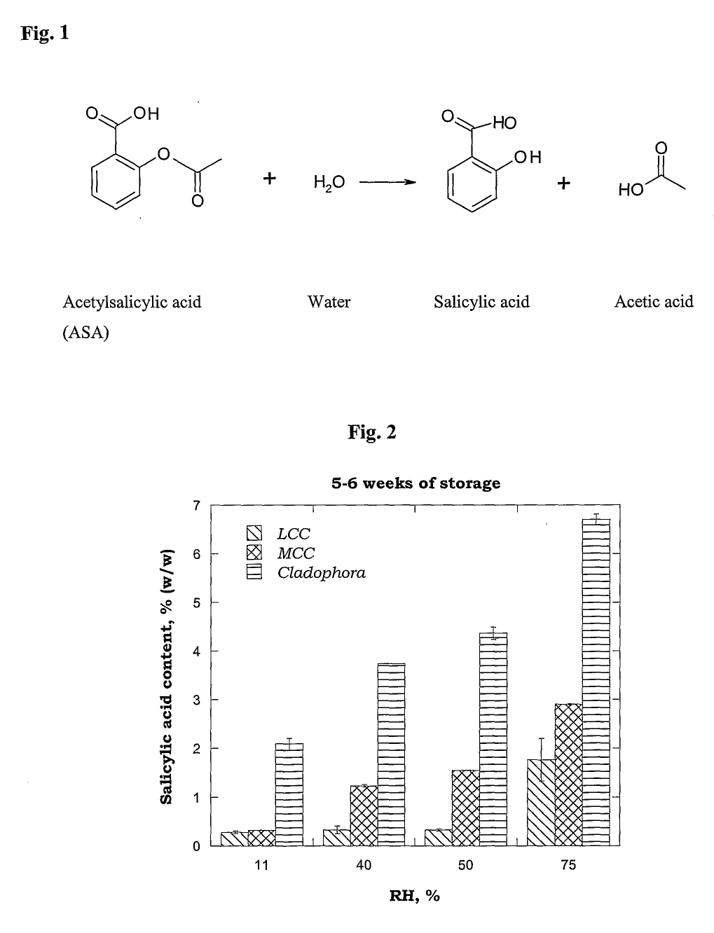

ASA (or aspirin) has been used as a model substance for stability studies for many years; the aspirin degradation in contact with various excipients has been studied. Upon contact with water, aspirin hydrolyzes to form salicylic acid and acetic acid, as shown in FIG. 1, and the substance is therefore suitable for a demonstration of the advantages of the present invention.

Quite surprisingly and contrary to the common practice of avoiding high-moisture-content hygroscopic materials in mixtures with moisture sensitive drugs, substantially higher stability is achieved for a model moisture sensitive drug, aspirin, in mixtures with a high-moisture-content hygroscopic low crystallinity cellulose powder as compared to the stability for mixtures with ordinary MCC. The powdered low crystallinity cellulose has excellent tabletting properties and it is stable.

A low crystalline cellulose to be used according to the invention has a crystallinity index less than about 75%, and in alternative embodiments, less than about 70%, less than about 67%, less than about 65%, less than about 63%, less than about 60%, less than about 57%, less than about 55%, less than about 53%, or less than about 50%, as measured with x-ray diffraction (XRD) according to the method outlined in Mihranyan et al (2004), cited above. In one embodiment, the LCC has a crystallinity index of from about 45% to about 50%, and more specifically about 48%.

Swelling of ordinary MCC, a suitable starting material for production of low crystallinity cellulose, induces several structural changes in the material.

1) The monolayer sorption capacity for water molecules, measured by extracting the BET area from water sorption isotherms, is considerably increased, for example by more than 40%. In one embodiment, this capacity is increased by around 100%. These values refer to room-temperature measurements.

ii) The hysteresis between water sorption and desorption isotherms is dramatically increased, for example by more than about 40%, and up to about 150% as achieved in some examples, in the area between the isotherms over the entire relative humidity range. The increase is even larger and above 80% if only the lower relative humidity range between about 10 and 60% is considered. In one embodiment, this increase is around 220% in the 10-60% RH range. These values refer to room-temperature measurements.

iii) The crystallinity index is considerably lowered, for example, by more than about 8%, 12%, 18%, 25%, 31% or 37%, as measured with XRD according to the method outlined in Mihranyan et al (2004), cited above, without more than only minor transition from a cellulose I to a cellulose II structure (i.e. mercerization). Thus, the low crystallinity cellulose is substantially non-mercerized.

iv) Compared to other swelling agents (e.g., some acids) the swelling procedure of the present invention does not impact considerably on the degree of polymerization (chain length), see e.g., Modi et al, J. Appl. Polymer Sci., 7, 15 (1963) and Neale, J. Textile Inst., Trans., 22, T320 (1931) or Shirley Inst. Mem., 10, 1 (1931).

v) Pore structure and corresponding surface area available for gas (e.g. N₂) sorption is diminished.

Among the parameters of the cellulose material to be used according to the invention, water monolayer sorption capacity (measured by extracting the BET area from water sorption isotherms), as well as the hysteresis area between water sorption and desorption isotherms, XRD and ¹³C CP/MAS NMR are of special importance.

By measuring the crystallinity index according to the method outlined in Mihranyan et al (2004) cited above, it has been found that, in one embodiment, the low crystallinity cellulose to be used according to the invention has a crystallinity index lower than about 75%, as measured with XRD, and preferably lower than about 60%, as measured with XRD. The ¹³C CP/MAS NMR spectrum of the low crystallinity cellulose to be used according to the invention differs from that of ordinary MCC in dry state but becomes similar to that of ordinary MCC when wetted with water.

Ordinary microcrystalline cellulose used in a number of pharmaceutical applications has a corresponding index around 80%, and typically between 82 and 85%, as measured with XRD according to the method outlined in Mihranyan et al (2004) cited above.

In one embodiment, a starting material for preparing the LCC is MCC, and the LCC is formed by swelling the MCC in a solution. The degree of polymerization (DP) for MCC products is generally in the range of from about 100 to about 300, and typically around 220. Swelling, in accordance with the present invention, introduces no or only negligible depolymerization, which means that the low crystallinity product has a DP of at least about 100, and, in one embodiment, in the same range of from about 100 to about 300 as that of the MCC starting material. When the starting material is MCC, this means that swelling will result in low crystallinity cellulose with a DP in a range proven highly beneficial for the tabletting process.

Other starting materials may alternatively be used where swelling is achieved at a different stage of the process and is combined with a depolymerization reaction, e.g. with acid to obtain a product with a desired DP of at least about 100, for example from about 100 to about 300. For example, the starting material may be purified alfa-cellulose of higher molecular weight than MCC and the process comprises swelling followed by depolymerization. Alternatively, depolymerization and swelling may be achieved in one step with, e.g., an acidic salt solution. Here, the reaction conditions need to be controlled so that depolymerization below a desired DP of about 100 is avoided. Typically, the LCC product will have a DP of from about 100 to about 600, more specifically from about 100 to about 300.

Alternatively, the starting material may be purified alfa-cellulose of higher molecular weight than powdered cellulose and the process comprises swelling followed by depolymerization by mechanical disintegration. Here, the mechanical disintegration of cellulose mass needs to be controlled so that a DP of from about 100 to about 600, more specifically about 100 to about 300, is achieved.

In one embodiment of the invention, swelling of a limited volume at the surface of the cellulose is achieved in order to provide the desired crystallinity index of the cellulose material as a surface characteristic. Here, the swelling agent needs to penetrate only a short distance into the structure with favorable reaction times. The crystallinity lowering treatment of the present invention then only needs to affect a thin surface layer, for example with a thickness of at least 10 nm, of the cellulose which physically contacts the moisture sensitive compound. If the crystallinity index is measured for the whole cellulose particle after such a treatment it will be larger than that for the essential portion of the surface. In this embodiment of the invention, it is the crystallinity index of the treated surface layer that should be considered. One of ordinary skill in the art can determine the appropriate concentrations and reaction times.

The swelling procedure of the present invention does not impact considerably on the degree of polymerization of the starting material (as do, e.g., certain types of acidic swelling agents). The swelling procedure induces small separations between the cellulose polymer chains, thus, reducing the overall cellulose crystallinity. The swelling does not cause the starting material to dissolve, and after washing, or similar treatments, toxic levels of impurities should not be present in the material. Various methods to induce the proper changes in the starting material, as outlined above, could be used. ZnCl₂ swelling is specifically described below to exemplify the invention. However, one of ordinary skill in the art will appreciate that the solution may be provided by any of a number of compounds including metal salts and the like. Examples of such salts include, but are not limited to, calcium and aluminum chloride; potassium, magnesium, calcium, strontium and barium iodide; calcium nitrate; sodium, calcium, zinc and aluminum thiocyanate; barium iodomercurate; sodium sulfide; and mixtures thereof. As well, various compounds such as different organic and inorganic bases below mercerizing strength, for example, amines or diamines, alkali, and the like, may be used as a swelling agent.

Appropriate concentrations of the compound, for example ZnCl₂ chosen to illustrate the invention below, inducing the proper crystallinity reduction in the starting material can be determined by one of ordinary skill in the art. In one embodiment, wherein the swelling agent comprises ZnCl₂, the concentration should be above about 55 wt./wt. %. In further embodiments, concentrations of from about 63 wt./wt. % to about 72 wt./wt. %, e.g. 70 wt./wt. % are employed. The results presented in FIGS. 2-4 as described in the examples below are based on a ZnCl₂ swollen cellulose manufactured as described with a 1 hour treatment of Avicel PH102 in a 70 wt./wt. % ZnCl₂ solution at room temperature. The cellulose used in the examples has a crystallinity index of about 48%.

As described above, one aspect of the invention is the use of the low crystallinity cellulose in the production of compositions comprising a pharmaceutically active agent, for example a moisture sensitive drug. Various moisture sensitive drugs are known in the art and are suitable for use in combination with the low crystallinity cellulose as described herein in the present pharmaceutical compositions. Examples of moisture sensitive drugs are substances containing chemical groups susceptible to hydrolysis, including, but not limited to, esters (acetylsalicylic acid, atropine, etc); lactams (penicillin G, etc); lactones (warfarin, etc); acetals and hemi-acetals (erythromycin, etc); carbamic esters (loratadine, etc); imides (barbiturates, etc); imines (diazepam, etc); amides (chloramphenicol, etc); alkyl halides (chlorambucil, etc), ketals and hemi-ketals, or phosphates and sulfate esters (see “Waterman et al. (2002) Hydrolysis in Pharmaceutical Formulations, Pharm. Dev. Tech., 7 (2), 113-146). Suitably, the pharmaceutical composition comprises a pharmaceutically effective amount of the active ingredient, in combination with the low crystallinity cellulose, optionally in combination with one or more additional conventional pharmaceutical ingredients. In specific embodiments, the pharmaceutical composition comprises from about 0.001 to about 90 wt. % of the active ingredient, or, alternatively, from 0.001 to about 80 wt. %, from 0.001 to about 70 wt. %, from 0.001 to about 60 wt. %, from 0.001 to about 50 wt. %, from 0.001 to about 40 wt. %, from 0.001 to about 30 wt. %, from 0.001 to about 20 wt. %, or from 0.001 to about 10 wt. %, of the active ingredient, and about 1 to about 99 wt. % of the low crystallinity cellulose.

The low crystallinity cellulose is advantageous in providing good tabletting properties. Accordingly, in one embodiment, the pharmaceutical composition is in tablet form, and may be formed using conventional tabletting procedures. There are also other situations where moisture sensitive agents need to be formulated with excipients of various types. Many tablet compositions require a granulated powder to be able to consolidate the active agent into good tablets. A further aspect is therefore the use of the LCC as an excipient for granulation whereby the composition is in the form of a granulate. The common dosage system for penicillines are as granulates for oral suspensions or mixtures. A great number of such compositions have been delivered to the market but it has been found to be difficult to obtain the desired three years stability with the MCC commonly used. Use of the low crystallinity cellulose in accordance with the present invention thus offers clear advantages also in the preparation of granulates for oral suspensions or mixtures.

The following examples demonstrate the preparation of low crystallinity cellulose for use in compositions according to the invention and demonstrate the advantages thereof for use with moisture sensitive active ingredients. As will be set forth in detail below, acetylsalicylic acid is used as an exemplary moisture sensitive drug in the examples. Moisture degradation of the acetylsalicylic acid results in salicylic acid, as shown in FIG. 1. Accordingly, the examples measure the amount of salicylic acid in the respective compositions, with an increasing amount of salicylic acid indicating a greater instability of the respective composition.

### EXAMPLE 1

In this example, the low crystallinity cellulose is prepared by swelling MCC in a solution of a metal salt to reduce the crystallinity of the cellulose. In this specific embodiment, the metal salt comprises zinc chloride (ZnCl₂), although one of ordinary skill in the art will appreciate that a number of other solutions may be employed. The manufacturing procedure of ZnCl₂ swollen-cellulose is outlined in Mihranyan et al (2004) cited above, and hereby incorporated herein by reference. In this example, 50 g of MCC is dispersed in 1 liter of 70 wt. % ZnCl₂ solution and vigorously stirred. After allowing the cellulose to swell for 1 hour, the cellulose is precipitated with additional water. The resultant powder is washed repeatedly until the conductivity of the wash water approximates that of deionized water (i.e. 10⁻⁶ S/cm) and, thereafter, a 10% suspension of the resultant powder (w/v) is spray-dried (Minor Type 53, Niro Atomizer A.S., Denmark) at Tᵢₙ=205-210° C. and Tₒᵤₜ=95-100° C. with a feed-rate of 1.7 l/h. The resulting material has a crystallinity index (CrI) of approximately 45% as measured with X-ray diffraction (XRD) according to the procedure as disclosed by Mihranyan et al. It has a large hysteresis between the water sorption and desorption isotherms, and during sorption its moisture content is as high as 3.4 and 12.1 wt./wt. % for relative humidity (RH) of approximately 11% and 75%, respectively. The term sorption, as used in the present specification, comprises both adsorption and absorption. The corresponding numbers for MCC (Avicel PH102) with a CrI of approximately 82% (measured with XRD) are 2.2 and 8.0 wt./wt. %, while for the high-crystallinity Cladophora cellulose having a CrI of approximately 95% (measured with XRD), the moisture content is as low as 1.2 and 6.4 wt./wt. % at a RH of 11% and 75%, respectively.

### EXAMPLE 2

This example tests the extent of moisture-induced degradation of the model substance acetylsalicylic acid (ASA), in contact with the different celluloses. From the above known facts, the high crystallinity Cladophora cellulose would appear to be an excellent candidate to the extra-dried excipient forms available on the market, due to its intrinsically low hygroscopicity, with MCC also being expected to provide good stability.

A physical mixture of 1 g of ASA and 3 g of each cellulose, namely ZnCl₂ swollen low crystallinity cellulose as described in Example 1, Avicel PH102, and Cladophora cellulose, are prepared using a Turbula mixer (Willy A. Bachofen AG, Switzerland) for 15 minutes. The drug-loaded samples are stored at 50° C. in dessicators with specified relative humidities of approximately 11, 40, 50, and 75% using saturated salt solutions. The salicylic acid content in the loaded powders is measured immediately after loading and prior to transfer of the loaded samples into dessicators as described below. The degradation of ASA is then examined with a UV-spectrophotometer (Hitachi U1100, Japan) by measuring the absorbance of salicylic acid in 95% ethanol. To conduct the measurement, sample/ethanol suspension is vigorously shaken and then centrifuged at 5000 rpm, 5 min (Eppendorf, Germany). The clear solution is pipetted out and subsequently used for the UV analysis.

FIG. 2 depicts the degradation pattern of ASA in the respective cellulose powders after 5 to 6 weeks of storage in the four different relative humidities of approximately 11%, 40%, 50% and 75% RH, at a temperature of 50° C. The ZnCl₂ swollen crystallinity cellulose is denoted LCC and Avicel PH102 is denoted MCC in FIG. 2. The low crystallinity cellulose was manufactured according to the description in Example 1 with a 1 hour treatment of Avicel PH102 in a 70 wt. % ZnCl₂ solution at room temperature.

From FIG. 2, it is observed that in the mixtures with the low crystallinity cellulose, the degradation of ASA is up to approximately 5 times lower than in the mixtures with ordinary MCC and up to approximately 13 times lower than in the mixtures with the low-hygroscopic Cladophora cellulose.

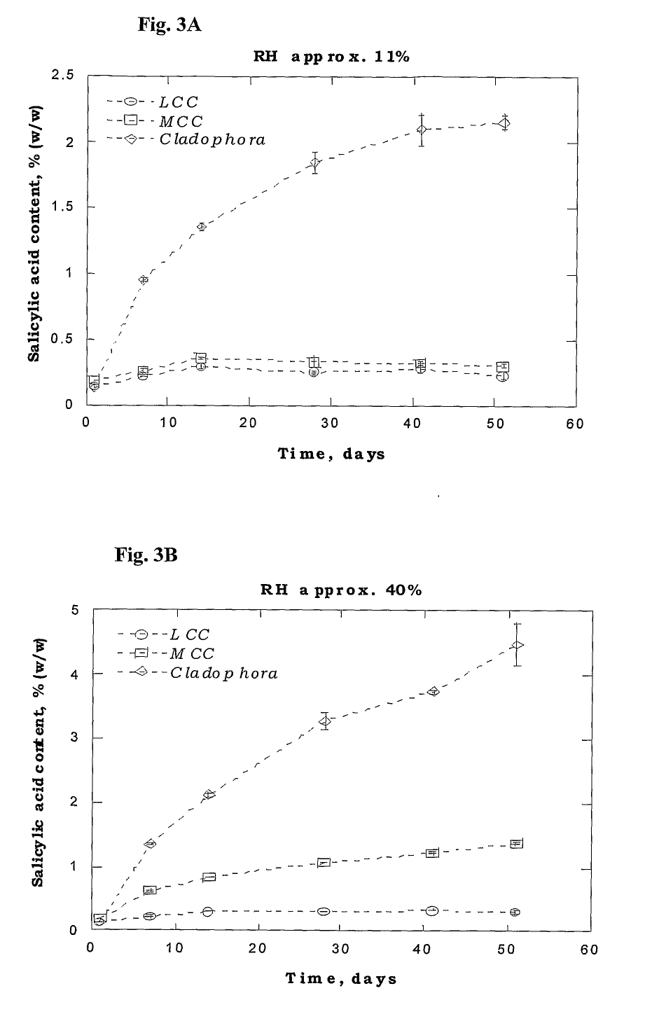

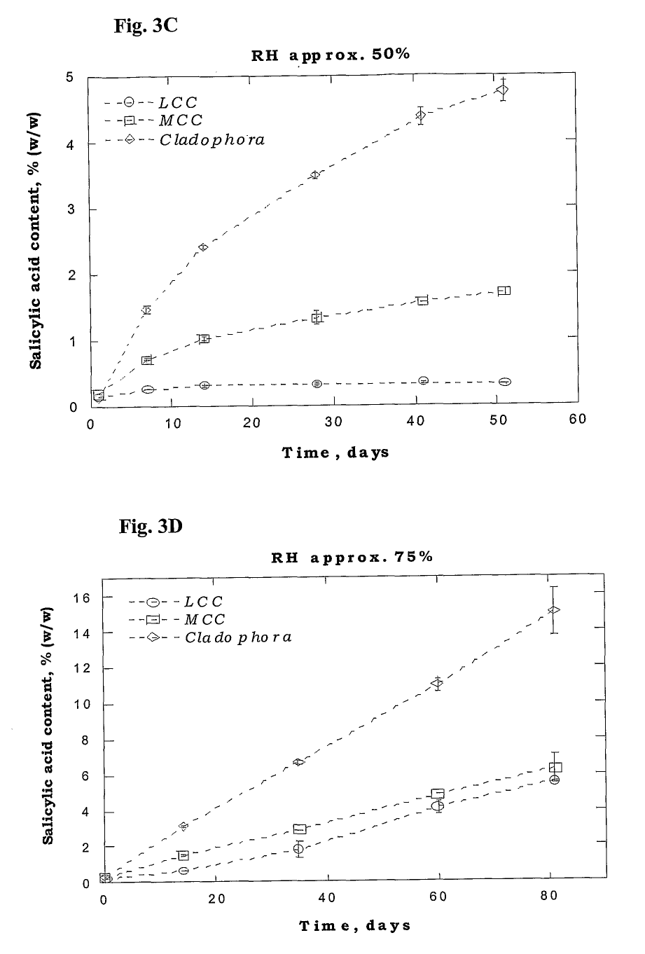

In FIGS. 3A-3D, the degradation of ASA can be followed in the three mixtures for the four different RHs of approximately 11%, 40%, 50% and 75% at a temperature of 50° C. From these results, it is observed that the moisture-sensitive ASA remains virtually intact in the mixtures with low crystallinity cellulose over the entire observation period of approximately 2 months at RHs of 11%, 40% and 50%. Even at RHs as high as 75%, the low crystallinity cellulose induces a lower undesired degradation than do the MCC and the low-hygroscopic Cladophora cellulose.

### EXAMPLE 3

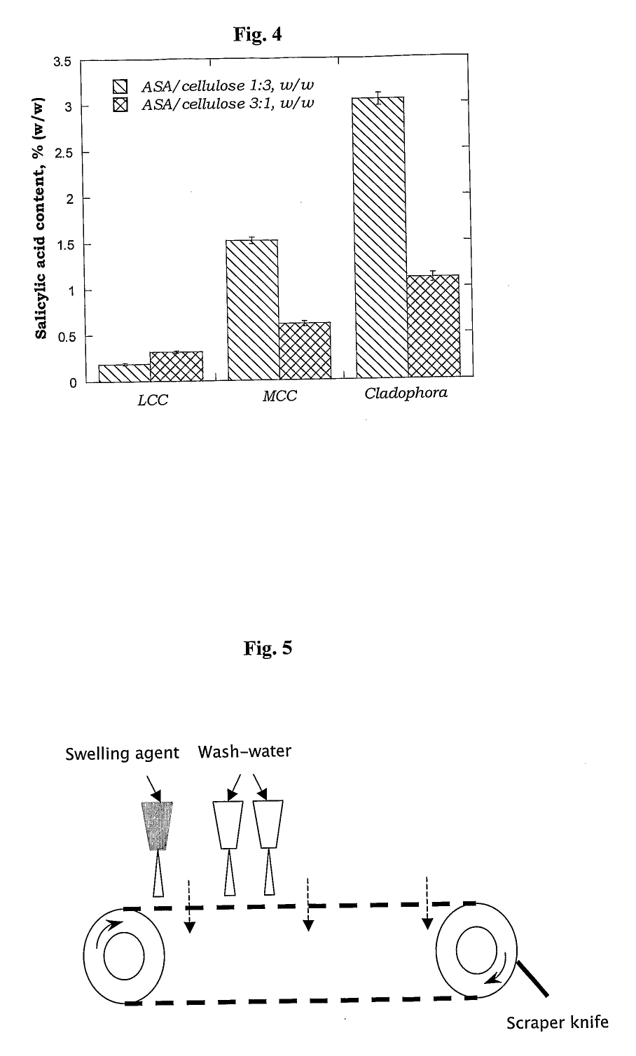

The stability of ASA is also tested in a closed vial experiment using different ASA/cellulose weight ratios. Compositions comprising 1:3 (wt./wt.) and 3:1 (wt./wt.) ASA/cellulose mixtures are sealed in 10 ml glass vials and stored at a temperature of 50° C. for 4 weeks. Under these conditions, the RH in the sealed glass vial is controlled by the moisture present inside the samples rather than externally imposed by saturated salt solutions as described in the previous example. The different ASA/cellulose ratios were chosen to mimic those situations when the amount of the moisture-sensitive drug substance prevails over that of cellulose and vice versa. From FIG. 4, it is observed that after 4 weeks of storage, the extent of ASA degradation was significantly lower in the ASA/LCC mixtures as compared to ASA/MCC and ASA/Cladophora mixtures.

### EXAMPLE 4

This example demonstrates another process for producing LCC for use in the invention. 500 g of dissolving pulp is milled in a hammer mill to a particle size smaller than 100 μm. The cellulose particles are sprayed with 500 g of 70 wt. % ZnCl₂ solution at room temperature and washed with about 25 liters of hot deionised water. After washing, a suspension with cellulose of 20 wt. % dry solids is spray-dried to 95 wt. % dry solids. The spray-died powder is fractionated through a 100 μm screen. The resulting powder, in mixtures with ASA, hampers the ASA degradation. The resulting powder has a much better tablettability than lactose.

### EXAMPLE 5

This example further demonstrates LCC for use according to the invention. 500 g of a cake is taken out just after the washing step following the hydrolysis in the production of conventional MCC. The moisture content is about 50 wt. %. To this cake, 834 g of solid milled fine powder of ZnCl₂ is added and the wet cake/ZnCl₂ is mixed in a high-speed mixer a very short time. After this, water is added in excess and the cellulose residue is washed until the conductivity of the wash water is below 3.5 μS/cm. The residue is diluted with water to about 20 wt. % dry solids and the resulting suspension is spray-dried. An excellent tabeletting powder is obtained and produces tablets of similar strength as ordinary commercially available MCC. The ¹³C CP/MAS NMR spectrum of the produced low crystallinity cellulose differs from ordinary MCC in dry state but is similar to that of ordinary MCC when wetted with water.

### EXAMPLE 6

This example further demonstrates LCC for use according to the invention. A thin layer of microcrystalline cellulose is placed on a perforated circulating band covered with a filter cloth so that water can be drained through it as illustrated in FIG. 5. A ZnCl₂ solution of suitable concentration serves as a swelling agent and is sprayed on top of the powder as the band is circulating to allow swelling. Upon swelling, the wet mass is sprayed with an excess of hot wash-water to remove traces of the swelling agent. The drainage may be aided by vacuum applied from the inner side of the circulating band. The cake is removed by a scraper knife at the end of the band. The velocity of the band rotation, the length of the circulating band, the swelling time, and the amount of wash-water is easily adjusted by one of skill in the art. The resultant cake is then spray-dried to provide an LCC suitable for use as described herein.

The specific examples and embodiments described herein are exemplary only in nature and are not intended to be limiting the invention defined by the claims. Further embodiments and examples will be apparent to one of ordinary skill in the art in view of the specification and are within the scope of the claimed invention.

What is claimed is:

1. A pharmaceutical composition comprising a pharmaceutically effective amount of an active ingredient and low crystallinity cellulose having a crystallinity index of less than about 75% as measured by X-ray diffraction and a degree of polymerization of at least about 100.

2. A pharmaceutical composition according to claim 1, wherein the active ingredient is moisture sensitive.

3. A pharmaceutical composition according to claim 1, wherein the active ingredient contains an ester (acetylsalicylic acid, atropine, etc), a lactam (penicillin G, etc), a lactone (warfarin, etc); an acetal and/or a hemi-acetal (erythromycin, etc), a carbamic ester (loratadine, etc), an imide (barbiturates, etc), an imine (diazepam, etc), an amide (chloramphenicol, etc); an alkyl halide (chlorambucil, etc), a ketal and/or a hemi-ketal, a phosphate and/or a sulfate ester.

4. A pharmaceutical composition according to claim 1, wherein the low crystallinity cellulose has a crystallinity index of less than about 65% as measured by X-ray diffraction.

5. A pharmaceutical composition according to claim 1, wherein the low crystallinity cellulose has a crystallinity index of less than about 55% as measured by X-ray diffraction.

6. A pharmaceutical composition according to claim 1, wherein the low crystallinity cellulose has a crystallinity index of from about 45% to about 55% as measured by X-ray diffraction.

7. A pharmaceutical composition according to claim 1, wherein the low crystallinity cellulose has a degree of polymerization of from about 100 to about 600.

8. A pharmaceutical composition according to claim 1, wherein the low crystallinity cellulose has a degree of polymerization of from about 100 to about 300.

9. A pharmaceutical composition according to claim 1, wherein the low crystallinity cellulose is produced by swelling microcrystalline cellulose in a solution.

10. A pharmaceutical composition according to claim 9, wherein the low crystallinity cellulose is produced by swelling microcrystalline cellulose in a solution comprising a metal salt.

11. A pharmaceutical composition according to claim 9, wherein the low crystallinity cellulose is produced by swelling microcrystalline cellulose in a solution comprising a metal chloride, a metal iodide, a thiocyanate, or a mixture of two or more thereof.

12. A pharmaceutical composition according to claim 9, wherein the low crystallinity cellulose is produced by swelling microcrystalline cellulose in a solution comprising zinc chloride.

13. A pharmaceutical composition according to claim 1, comprising from about 0.001 to about 90 wt. percent of the active ingredient and from about 1 to about 99 wt. percent of the low crystallinity cellulose.

14. A pharmaceutical composition according to claim 1, in tablet form.

15. A pharmaceutical composition according to claim 1, in granulate form.

16. A pharmaceutical composition according to claim 2, wherein the low crystallinity cellulose is produced by swelling microcrystalline cellulose in a solution.

17. A pharmaceutical composition according to claim 2, wherein the low crystallinity cellulose is produced by swelling microcrystalline cellulose in a solution comprising zinc chloride.

18. A pharmaceutical composition according to claim 2, wherein the low crystallinity cellulose has a degree of polymerization of from about 100 to about 600.

19. A pharmaceutical composition according to claim 2, comprising from about 0.01 to about 90 wt. percent of the active ingredient and from about 1 to about 99 wt. percent of the low crystallinity cellulose.

20. A pharmaceutical composition according to claim 2, in tablet form.

21. A pharmaceutical composition according to claim 2, in granulate form.
